(19) United States
(12) Patent Application Publication
(10) Pub. No.: US 2023/0010769 A1
(43) Pub. Date: Jan. 12, 2023

(54) INFORMATION PROCESSING SYSTEM, INFORMATION PROCESSING APPARATUS, INFORMATION PROCESSING METHOD, AND NON-TRANSITORY STORAGE MEDIUM

(71) Applicant: CANON KABUSHIKI KAISHA, Tokyo (JP)

(72) Inventors: Kohtaro Umezawa, Tokyo (JP); Kojiro Nishi, Kanagawa (JP); Ryuta Ueda, Kanagawa (JP)

(21) Appl. No.: 17/858,475

(22) Filed: Jul. 6, 2022

(30) Foreign Application Priority Data
Jul. 7, 2021 (JP) 2021-112966

(51) Int. Cl. G16H 50/20; G06N 5/04

(52) U.S. Cl. CPC G16H 50/20; G06N 5/04

(57) ABSTRACT

An information processing system includes a first information processing apparatus including a first inference unit configured to perform first inference processing on inference target medical data using a first partial model including an input layer and at least some of intermediate layers and corresponding to a plurality of second partial models, and a first output unit configured to output a result of the first inference processing and selection information to a second information processing apparatus, and the second information processing apparatus including a second inference unit configured to perform second inference processing by inputting a result of the first inference processing to a second partial model selected from among the plurality of second partial models based on the selection information.

### BACKGROUND

### Field of the Disclosure

The present disclosure relates to an information processing system that performs inference processing using an inference model, an information processing apparatus, and an information processing method, and a non-transitory storage medium.

### Description of the Related Art

A system that performs inference processing (disease detection, benign and malignant differentiation, prognostic prediction, risk prediction, etc.) on a predetermined disease by applying a machine learning technique to medical data, including a medical image captured by a medical imaging device (modality) and medical information acquired from a medical information system, is known.

Zihao Liu et al., “Orchestrating Medical Image Compression and Remote Segmentation Networks”, International Conference on Medical Image Computing and Computer-Assisted Intervention—MICCAI 2020 discusses a technique in which, when an image is input, image compression is performed by a first information processing apparatus that is a local information processing apparatus, and the compression result is transferred to a second information processing apparatus located on a remote cloud service to perform disease image segmentation.

Behzad Bozorgtabar et al., “SALAD: Self-Supervised Aggregation Learning for Anomaly Detection on X-Rays”, International Conference on Medical Image Computing and Computer-Assisted Intervention—MICCAI 2020 discusses a technique for learning a model composed of an encoder and a decoder using a medical image as an input and extracting features using an inference model (encoder unit).

In the technique discussed by Zihao Liu et al., “Orchestrating Medical Image Compression and Remote Segmentation Networks”, International Conference on Medical Image Computing and Computer-Assisted Intervention—MICCAI 2020, the medical image is transmitted to the cloud service as inference target data to perform segmentation of the medical image. In this case, the inference target data, such as medical data, is transmitted to an external information processing apparatus from within a hospital. This makes it difficult to protect the privacy of the inference target data.

On the other hand, as in the technique discussed by Behzad Bozorgtabar et. al., “SALAD: Self-Supervised Aggregation Learning for Anomaly Detection on X-Rays”, International Conference on Medical Image Computing and Computer-Assisted Intervention—MICCAI 2020, an inference model can be built in an information processing apparatus managed by a user who executes inference processing. In this case, it is difficult to prevent abuse of the inference model. For example, it is difficult to prevent the user from copying the inference model and distributing copies of the inference model to a third party, or altering the inference model. This leads to difficulty in securing the confidentiality of the inference models.

### SUMMARY

The present disclosure is directed to providing an information processing system, an information processing method, and a non-transitory storage medium to secure the confidentiality of an inference model for performing inference processing on inference target data while protecting the privacy of the inference target data.

According to an aspect of the present invention, an information processing system includes a first information processing apparatus, and a second information processing apparatus configured to communicate with the first information processing apparatus via a network, wherein the first information processing apparatus includes a first acquisition unit configured to acquire inference target medical data and selection information indicating a partial model to be applied to the inference target medical data, a first inference unit configured to perform first partial inference processing on the inference target medical data using a first partial model, the first partial model including an input layer and at least some of intermediate layers of a neural network, wherein a plurality of second partial models includes the intermediate layers not included in the first partial model, and the neural network includes the input layer, the intermediate layers, and an output layer, and a first output unit configured to output a result of the first inference processing and the selection information to the second information processing apparatus, and wherein the second information processing apparatus includes a second acquisition unit configured to acquire the result of the first inference processing and the selection information from the first information processing apparatus, and a second inference unit configured to perform second inference processing by inputting the result of the first inference processing to a second partial model selected from among the plurality of second partial models based on the selection information.

Further features of the present invention will become apparent from the following description of exemplary embodiments with reference to the attached drawings.

### BRIEF DESCRIPTION OF THE DRAWINGS

FIG. 1 is a block diagram illustrating a configuration of an information processing system according to a first exemplary embodiment.

FIG. 2 is a schematic diagram illustrating an inference model according to the first exemplary embodiment.

FIG. 3 is a block diagram illustrating a hardware configuration of a first information processing apparatus according to the first exemplary embodiment.

FIG. 4 is a flowchart illustrating inference processing to be performed by the information processing system according to the first exemplary embodiment.

FIG. 5 is a flowchart illustrating a modified example of inference processing to be performed by an information processing system according to a second exemplary embodiment.

FIG. 6 is a block diagram illustrating a configuration of an information processing system according to a third exemplary embodiment.

FIG. 7 is a schematic diagram illustrating an inference model according to the third exemplary embodiment.

FIG. 8 is a flowchart illustrating inference processing to be performed by the information processing system according to the third exemplary embodiment.

FIG. 9 is a block diagram illustrating a configuration of an information processing system according to a fourth exemplary embodiment.

FIG. 10 is a schematic diagram illustrating an inference model according to the fourth exemplary embodiment.

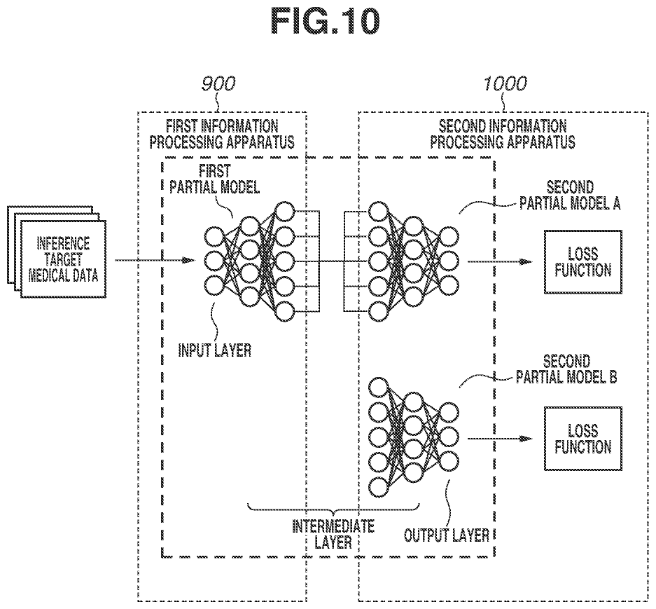

FIG. 11 is a flowchart illustrating training processing to be performed by the information processing system according to the fourth exemplary embodiment.

FIG. 12 is a block diagram illustrating a configuration of an information processing system according to a fifth exemplary embodiment.

FIG. 13 is a schematic diagram illustrating an inference model according to the fifth exemplary embodiment.

FIG. 14 is a flowchart illustrating training processing to be performed by the information processing system according to the fifth exemplary embodiment.

### DESCRIPTION OF THE EMBODIMENTS

The present disclosure can be suitably applied to raw data (signal data) acquired using a modality and medical data such as medical data for diagnosis generated by image reconfiguration based on raw data.

Examples of the modality include an X-ray computed tomography (CT) apparatus, a magnetic resonance imaging (MRI) apparatus, a single-photon emission computed tomography (SPECT) apparatus, a positron emission tomography (PET) apparatus, and a cardiograph. Not only medical data, but also information related to the privacy of patients, such as information about age, gender, and disease information, can be used as inference target data and training data.

An inference process using an inference model in an information processing system according to the present invention will be described below in first to third exemplary embodiments. A training process using an inference model in the information processing system according to the present invention will be described below in fourth and fifth exemplary embodiments. As described below, the inference model used in the inference process is not limited to the inference model generated through the training process according to the fourth and fifth exemplary embodiments of the present invention. The inference model used in the inference process is a trained inference model trained by a known method or by performing training based on machine training or deep training in the training process according to the present invention. Any trained inference model may be used as long as the inference model is obtained by performing the training process so that predetermined conditions are satisfied. The trained inference model may also be used as a target for additional training, transfer training, fine-tuning, or the like. Therefore, training processing may be performed by the training process to be described below as additional training using the trained inference model trained by a known method, or training processing may be performed in a reverse procedure.

Exemplary embodiments of the present invention will be described below with reference to the accompanying drawings.

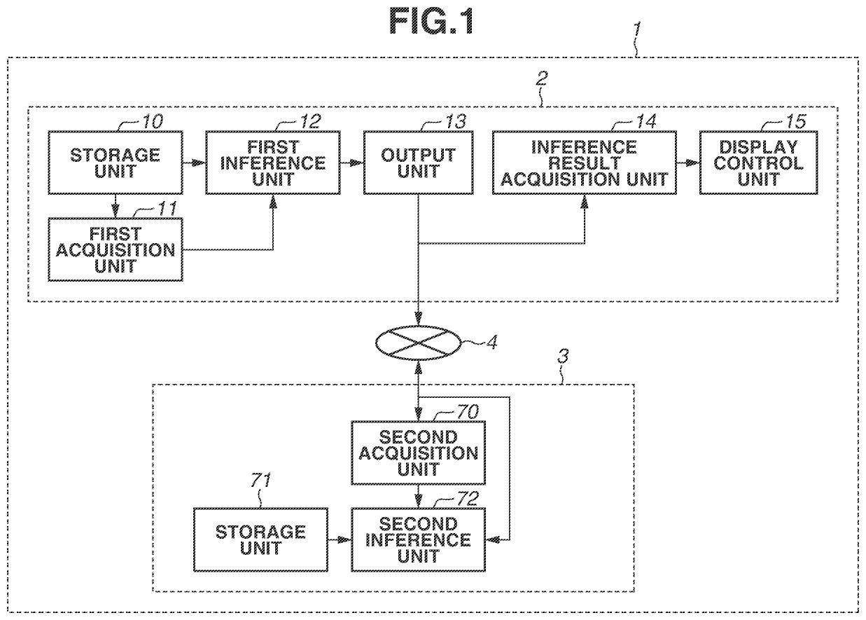

The first exemplary embodiment will now be described. An information processing system 1 according to the first exemplary embodiment will be described with reference to FIG. 1. The information processing system 1 according to the first exemplary embodiment includes a first information processing apparatus 2, a second information processing apparatus 3, and a network 4 that interconnects the first information processing apparatus 2 and the second information processing apparatus 3 in a communicable manner.

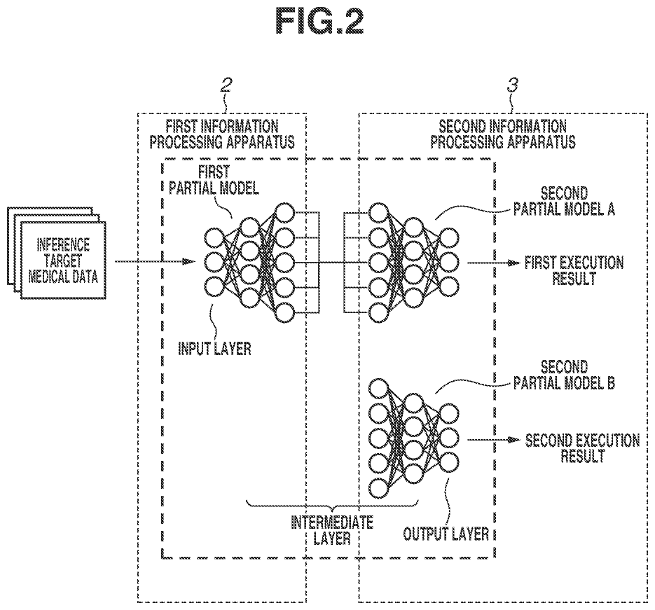

A configuration example of the information processing system 1 according to the first exemplary embodiment will now be described with reference to FIG. 2. The information processing system 1 includes the first information processing apparatus 2 and the second information processing apparatus 3. The first information processing apparatus 2 and the second information processing apparatus 3 each include a partial model as a part of an inference model to perform inference processing on inference target medical data and output an execution result. The second information processing apparatus 3 includes a plurality of partial models. The partial models have different layers following the layers forming the partial models in the first information processing apparatus 2, different parameters, and the like, and the partial models are obtained by performing training processing to classify different tasks and classes. The partial model in the first information processing apparatus 2 is a partial model corresponding to two or more partial models in the subsequent partial models. The term “inference model” used herein refers to a trained model based on a neural network including an input layer, intermediate layers, and an output layer. A parameter for outputting an inference result is determined in training processing, and a model obtained by pairing the parameter with a network model is defined as the inference model.

The first information processing apparatus 2 executes first inference processing using a first partial model including the input layer and at least some of the intermediate layers in the above-described trained inference model. The second information processing apparatus 3 executes second inference processing using a second partial model that matches selection information in a plurality of second partial models each formed of a layer different from the layers forming the first partial model in the above-described trained inference model. A configuration example of each of the first information processing apparatus 2 and the second information processing apparatus 3 will be described below.

The first information processing apparatus 2 is an information processing apparatus that can be operated by a user of the inference model. The user is authorized to manage the inference target medical data. The user is, for example, a healthcare worker. On the other hand, the second information processing apparatus 3 is an information processing apparatus owned by a provider of the model. The provider is authorized to manage the inference model used for inference processing. The second information processing apparatus 3 is located on an external server outside the first information processing apparatus 2 and is configured in a communicable manner via the network 4.

The first information processing apparatus 2 includes a first acquisition unit 11 that acquires inference target medical data and selection information indicating a plurality of selected partial models to be applied to the inference target medical data. The first information processing apparatus 2 also includes a first inference unit 12 that performs the first inference processing on the inference target medical data using the first partial model. The first partial model includes the input layer and at least some of the intermediate layers of the trained inference model based on the neural network including the input layer, the intermediate layers, and the output layer to perform inference processing on medical data, and corresponds to the plurality of second partial models formed of layers different from the layers forming the first partial model. In this case, each of the second partial models is a trained partial model trained by performing training processing on a specific trained inference model while fixing the parameter for the first partial model. Examples of the specific trained inference model include a trained inference model generated by training the inference model trained to classify the classes correlated with the plurality of second partial models or any one of the plurality of second partial models. The selection information indicating the selected second partial models is set such that the user inputs information corresponding to information about the classes classified by the respective partial models.

The selection information may be set by simply selecting or inputting the class on which the user intends to perform inference processing. Alternatively, an application order or the like of a plurality of partial models to be applied to inference processing may be set, and selection information indicating a plurality of selected partial models may be set based on the application order when the user inputs or selects some inference classes or inference tasks.

The first information processing apparatus 2 also includes an output unit 13 that outputs a first inference result obtained by the first inference processing and the selection information acquired by the first acquisition unit 11 to the second information processing apparatus 3. The second information processing apparatus 3 includes a plurality of second partial models formed of layers different from the layers forming the first partial model. The first information processing apparatus 2 also includes a storage unit 10 that stores the first partial model as a part of the trained inference model and the inference target medical data. The first information processing apparatus 2 also includes an inference result acquisition unit 14 and a display control unit 15. The inference result acquisition unit 14 acquires a second inference result from the second information processing apparatus 3, which is another information processing apparatus. The display control unit 15 causes a display device to display the acquired inference result.

### (Storage Unit 10)

The storage unit 10 stores the first partial model including the input layer of the trained inference model and the inference target medical data. The storage unit 10 stores, as the first partial model, a network corresponding to the partial model in association with a trained parameter corresponding to the network. The inference target medical data may be medical data automatically transferred from a modality or an external image server. The term “a part of the trained inference model” refers to a continuous portion ranging from a certain layer to another layer, but is not limited to this portion. A part of the trained inference model may be a continuous portion ranging from a certain neuron to another neuron, or may be an isolated neuron. Each partial model may be a plurality of portions that are not adjacent to each other in the trained inference model.

### (First Acquisition Unit 11)

The first acquisition unit 11 acquires the inference target medical data from the storage unit 10, and further acquires the selection information indicating a plurality of second partial models to be applied to the inference target medical data. The acquired inference target medical data and the acquired selection information are transmitted to the first inference unit 12.

### (First Inference Unit 12)

The first inference unit 12 acquires the first partial model from the storage unit 10, and performs the first inference processing using the first partial model on the inference target medical data. The first inference unit 12 transmits the first inference result based on the first partial model to the output unit 13. In the present exemplary embodiment, the first partial model includes the input layer and at least some of the intermediate layers of the trained inference model and corresponds to the plurality of second partial models in the second information processing apparatus 3. The output from the intermediate layers is transmitted to the output unit 13. In this case, the output from the intermediate layers is tensor information. If the inference model is a model based on a cellular neural network (CNN), the output is a feature map.

### (Output Unit 13)

The output unit 13 transmits the first inference result and the selection information to the second information processing apparatus 3, which is another information processing apparatus. In this case, the selection information is information indicating the plurality of second partial models to be applied, or information including the application order of the plurality of second partial models.

### (Inference Result Acquisition Unit 14)

The inference result acquisition unit 14 acquires the second inference result on the inference target medical data from the second information processing apparatus 3. Upon acquiring the inference result, the inference result acquisition unit 14 transmits the inference result to the display control unit 15.

### (Display Control Unit 15)

The display control unit 15 controls the display device to display the inference result acquired by the inference result acquisition unit 14. The display device is, for example, a display attached to an information processing apparatus or a mobile terminal that can be operated by a hospital official via an external server. The first information processing apparatus 2 may be composed of a computer including a processor, a memory, and a storage. In this case, programs stored in the storage are loaded into the memory and the programs are executed by the processor to thereby implement the functions and processing of the storage unit 10, the first acquisition unit 11, the first inference unit 12, the output unit 13, the inference result acquisition unit 14, the display control unit 15, and the like. However, the configuration of the first information processing apparatus 2 is not limited to this example. For example, a part of or the entire configuration of the first information processing apparatus 2 may be implemented by an exclusively designed processor (such as an application specific integrated circuit (ASIC)) or a field programmable gate array (FPGA). Alternatively, a part of arithmetic processing may be executed by a processor such as a graphics processing unit (GPU) or a digital signal processor (DSP). The first information processing apparatus 2 may be composed of a single piece of hardware, or may be composed of a plurality of pieces of hardware.

For example, the function and processing of the first information processing apparatus 2 may be implemented by a plurality of computers in cooperation using cloud computing or distributed computing.

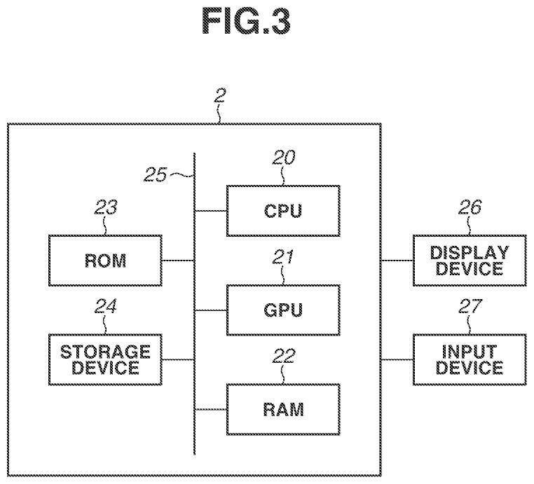

FIG. 3 is a block diagram illustrating a specific configuration example of the first information processing apparatus 2. In this example, the first information processing apparatus 2 includes a central processing unit (CPU) 20, a GPU 21, a random access memory (RAM) 22, a read-only memory (ROM) 23, and a storage device 24. The CPU 20, the GPU 21, the RAM 22, the ROM 23, and the storage device 24 are interconnected via a system bus 25. The first information processing apparatus 2 is also connected to each of a display device 26 and an input device 27 such as a mouse or a keyboard.

The first information processing apparatus 2 configured as described above enables the user who uses the inference model and manages medical data to obtain a plurality of inference results, as needed, while protecting the privacy of the medical data without the need of transmitting the inference target medical data to an external information processing apparatus. Even in a case where the user intends to obtain a plurality of inference results, the use of the first partial model as the partial model corresponding to the plurality of second partial models makes it possible to reduce the number of times of communication between the information processing apparatuses 2 and 3, reduce the data capacity, and reduce the number of requests to machine resources by performing a part of inference processing using another information processing apparatus.

The provider of the inference model incorporates only a part of the inference model into the first information processing apparatus 2, thereby securing the confidentiality of the inference model. In addition, the provider of the inference model provides the user with only the partial model corresponding to the plurality of second partial models, thereby securing the confidentiality regarding the network structure and parameters for the second partial models.

In this case, the second information processing apparatus 3 is located on an external server outside the first information processing apparatus 2, and includes a second acquisition unit 70 that acquires the first inference result from the first information processing apparatus 2 and the selection information indicating the plurality of second partial models to be applied. The second information processing apparatus 3 also includes a second inference unit 72 that performs second inference processing using the plurality of second partial models formed of layers different from the layers forming the first partial model in the inference processing based on the input first inference result in the trained inference model corresponding to the inference target medical data using the first partial model including the input layer and at least some of the intermediate layers in the trained inference model based on the neural network including the input layer, the intermediate layers, and the output layer to perform inference processing on medical data. The second information processing apparatus 3 also includes a storage unit 71 that stores the second partial models.

In the present exemplary embodiment, each of the plurality of second partial models includes intermediate layers different from the intermediate layers forming the first inference model and the output layer in the trained inference model.

The second partial models are partial models in which at least one of an inference task to be performed by each partial model and the inference class of each partial model is different from that of another partial model. The second partial models may have different network configurations, respectively, in view of properties such as a class.

### (Storage Unit 71)

The storage unit 71 stores networks corresponding to the plurality of second partial models, respectively, in association with trained parameters corresponding to the networks, respectively. Each partial model indicates a continuous portion ranging from a certain layer to another layer. However, the configuration of each partial model is not limited to this example. For example, each partial model may be a continuous portion ranging from a certain neuron to another neuron, an isolated neuron, or a plurality of portions that are not adjacent to each other in the inference model.

### (Second Inference Unit 72)

The second inference unit 72 acquires the second partial models that match the selection information from the storage unit 71, and performs the second inference processing using the second partial models on the inference target medical data. The second inference result is transmitted to the first information processing apparatus 2. In this case, if the selection information includes a plurality of second partial models, the second inference processing based on the input first inference result is performed on the plurality of second partial models. If the selection information includes the application order of the second partial models, the inference processing is performed in accordance with the application order. Upon receiving an inference result based on an i-th second partial model to be applied, the second inference unit 72 may determine whether to perform inference processing based on an (i+1)th second partial model. The second partial models selected based on the selection information are partial models for classifying a group of correlated classes. The inference task to be performed by each second partial model or the inference class of each second partial model is different from that of another second partial model. Examples of the second partial models include second partial models for determining the presence or absence of a node, second partial models for detecting a node, and second partial models for extracting a nodular area. If the degree of difficulty in inference processing for classification, detection, and extraction varies, the user may set selection information to determine the application order depending on the degree of difficulty in inference processing. The second partial models may be configured to have different numbers of layers forming the network, different depths of the layers, and the like. In the present exemplary embodiment, the plurality of second partial models each includes the output layer, and thus a plurality of inference processing results is output to the second information processing apparatus 3. On the other hand, the plurality of second partial models may be composed only of the intermediate layers and the output from the intermediate layers may be transmitted to the first information processing apparatus 2.

The second information processing apparatus 3 configured as described above enables the provider of the inference model to incorporate only a part of the trained inference model into the first information processing apparatus 2 and to store a part of the trained inference model in the second information processing apparatus 3 that is owned and managed by the provider. If the output layer is included in the second information processing apparatus 3, abuse of the inference model including the second partial models can be detected in consideration of the output from the output layer and the like. The provision of a plurality of second partial models corresponding to the first partial model enables the user to reduce the time and labor for creating input data for each inference model. The information processing system 1 described above enables the user to obtain a plurality of inference results, as needed, while protecting the privacy of the inference target medical data and securing the confidentiality of the inference models based on which inference processing is performed on the inference target medical data.

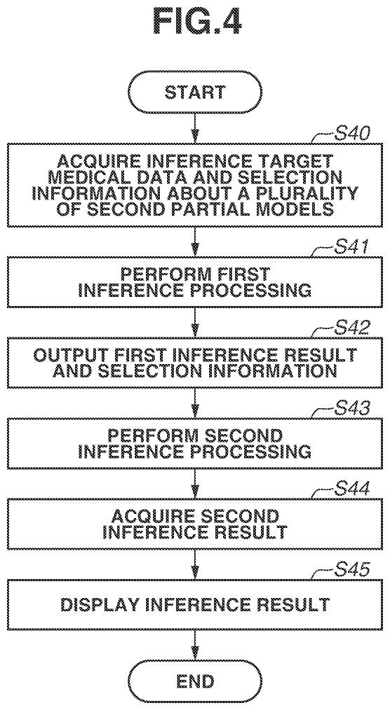

The inference process to be performed by the information processing system 1 according to the present exemplary embodiment will now be described with reference to FIG. 4.

In step S40, the first acquisition unit 11 in the first information processing apparatus 2 acquires inference target medical data and selection information indicating a plurality of second partial models. Upon acquiring the inference target medical data and the selection information indicating the plurality of second partial models, the first acquisition unit 11 transmits the acquired inference target medical data to the first inference unit 12. Then, the processing proceeds to step S41.

In step S41, the first inference unit 12 in the first information processing apparatus 2 executes first inference processing on inference target data using the first partial model that includes the input layer and at least some of the intermediate layers and corresponds to the plurality of second partial models formed of layers different from the layers forming the first partial model. After execution of the first inference processing, the first inference unit 12 transmits the inference result of the first inference processing and the selection information indicating the plurality of second partial models to the output unit 13. Then, the processing proceeds to step S42.

In step S42, the output unit 13 in the first information processing apparatus 2 outputs the inference result of the first inference processing and the selection information indicating the plurality of second partial models to the second information processing apparatus 3. Then, the processing proceeds to step S43.

In step S43, the second inference unit 72 in the second information processing apparatus 3 performs second inference processing using the plurality of second partial models formed of layers different from the layers forming the first partial model. In the present exemplary embodiment, each of the second partial models includes intermediate layers different from the intermediate layers forming the first partial model in the inference model. Each of the second partial models further includes the output layer, and outputs the inference result corresponding to the inference target data. The second partial models are partial models in which at least one of the inference class of each partial model and the inference task to be performed by each partial model is different from that of another partial model. In this case, each of the plurality of second partial models is a trained partial model trained by performing training processing on a specific trained inference model while fixing the parameter for the first partial model. Examples of the specific trained inference model include a trained inference model generated by training the inference model trained to classify the classes correlated with the plurality of second partial models, or by training any one of the plurality of second partial models. The second inference unit 72 transmits the inference result corresponding to the inference target medical data to the first information processing apparatus 2, and then the processing proceeds to step S44. If the inference processing is performed using a plurality of second partial models indicated by the selection information, the second inference unit 72 transmits a plurality of inference results to the first information processing apparatus 2. The second inference unit 72 may store the inference result of the second inference processing in the storage unit 71 and may present the inference result in response to an access from an external apparatus.

The output destination of the inference result is not limited to the first information processing apparatus 2. The inference result may be transmitted to a designated information terminal or contact address.

In step S44, the inference result acquisition unit 14 in the first information processing apparatus 2 acquires the second inference result as the inference results based on the plurality of second partial models from the second inference unit 72 in the second information processing apparatus 3. Upon acquiring the second inference result, the inference result acquisition unit 14 transmits the second inference result to the display control unit 15. Then, the processing proceeds to step S45.

In step S45, the display control unit 15 causes the display device 26 to display the inference result corresponding to the inference target medical data. The inference result to be displayed on the display device 26 is obtained by performing a series of inference processing (first inference processing and second inference processing) on the inference target medical data by the first information processing apparatus 2 and the second information processing apparatus 3. If a plurality of inference results based on the second partial models is obtained, the display control unit 15 displays the plurality of inference results in a contrastable manner. For example, the display control unit 15 may arrange and display the plurality of inference results, or may display the plurality of inference results by switching the inference results. Alternatively, the display control unit 15 may display a combined image obtained by superimposing the inference results on one image. More alternatively, the display control unit 15 may display the inference target medical data in association with the inference results, or may display information or the like about the inference model used for the inference processing.

Accordingly, the information processing system 1 according to the present exemplary embodiment can perform inference processing using a plurality of inference models, as needed, with low calculation cost while protecting the privacy of inference target medical data and securing the confidentiality of the inference models for performing inference processing on the inference target medical data. Since the first partial model corresponds to the plurality of second partial models, the time and labor for selecting a plurality of inference models to obtain a plurality of inference results can be reduced and the time and labor for processing inference data into a form that matches the inference models can also be reduced.

### Modified Example of First Exemplary Embodiment

The first exemplary embodiment described above illustrates an example where the second inference unit 72 performs inference processing using a plurality of second partial models that match selection information indicating the plurality of second partial models based on the selection information. The first exemplary embodiment described above also illustrates an example where the second inference unit 72 performs inference processing using the second partial models in accordance with the application order of the second partial models included in the selection information. In a modified example of the first exemplary embodiment, the inference result based on the i-th second partial model is input to the (i+1)th second partial model based on the application order. An example where a brain tumor area is roughly extracted in the i-th second partial model and then the brain tumor area is more accurately extracted in the (i+1)th second partial model will be described. The brain tumor area extracted in the i-th second partial model is input to the (i+1)th second partial model as reference information, thereby increasing the accuracy of extracting the brain tumor area in the (i+1)th second partial model.

With the configuration according to the modified example of the first exemplary embodiment, in addition to the advantageous effects obtained by the above-described exemplary embodiment, the inference processing can be performed with a higher accuracy by combining the inference results based on the plurality of second partial models.

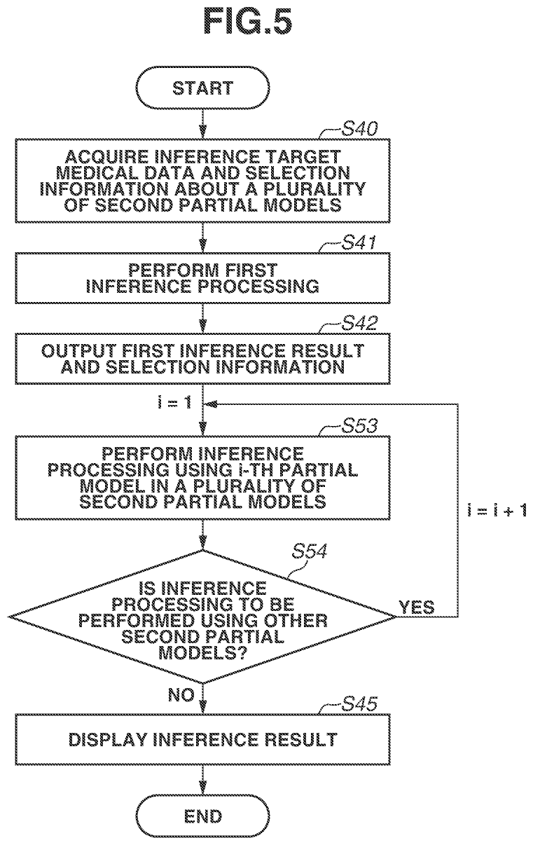

The first exemplary embodiment described above illustrates an example where the second inference unit 72 performs inference processing using a plurality of second partial models that match selection information indicating the plurality of second partial models based on the selection information. The first exemplary embodiment described above also illustrates an example where the second inference unit 72 performs inference processing using the second partial models in accordance with the application order of the second partial models included in the selection information. In the second exemplary embodiment, the second inference unit 72 further determines whether to perform inference processing using the (i+1)th second partial model based on the inference result based on the i-th second partial model having a higher application order. As a result of determination, if the second inference unit 72 determines not to perform inference processing using the (i+1)th second partial model, the inference processing using the (i+1)th second partial model is not executed. With this configuration, the calculation cost can be reduced and the time and labor for the user to compare the plurality of inference results can also be reduced. A processing flow of the information processing system 1 according to the second exemplary embodiment will be described below with reference to FIG. 5. Like in the above-described exemplary embodiment, the description of the processing flow is omitted as appropriate.

In In step S53, the second acquisition unit 70 acquires the first inference result based on the first partial model and the selection information indicating the plurality of second partial models from the first information processing apparatus 2. The second acquisition unit 70 transmits the acquired first inference result and the acquired selection information to the second inference unit 72. The second inference unit 72 determines the inference order of the second partial models in accordance with the application order of the second partial models included in the acquired selection information, and performs inference processing using the i-th second partial model.

In step S54, upon reception of the inference processing result based on the i-th second partial model, the second inference unit 72 determines whether to perform inference processing using the (i+1)th partial model and subsequent partial models. If the inference result based on the i-th second partial model is associated with inference processing using the (i+1)th partial model and subsequent partial models, the second inference unit 72 determines not to perform inference processing using another partial model based on the result of the i-th partial model. An example where the second partial models included in the selection information are partial models for classification, detection, and extraction of specific classes, respectively, will be described. In this case, if it is determined that there is no specific class based on the second partial model for classification, it is determined that inference processing using the partial models for detection and extraction is not performed. If it is determined that there is no second partial model based on which inference processing is to be performed, the processing proceeds to step S45. Then, this processing flow ends.

With the configuration according to the present exemplary embodiment, in addition to the advantageous effects obtained by the above-described exemplary embodiment, the advantageous effect of performing inference processing using appropriate partial models can be obtained even when a plurality of second partial models is selected based on selection information.

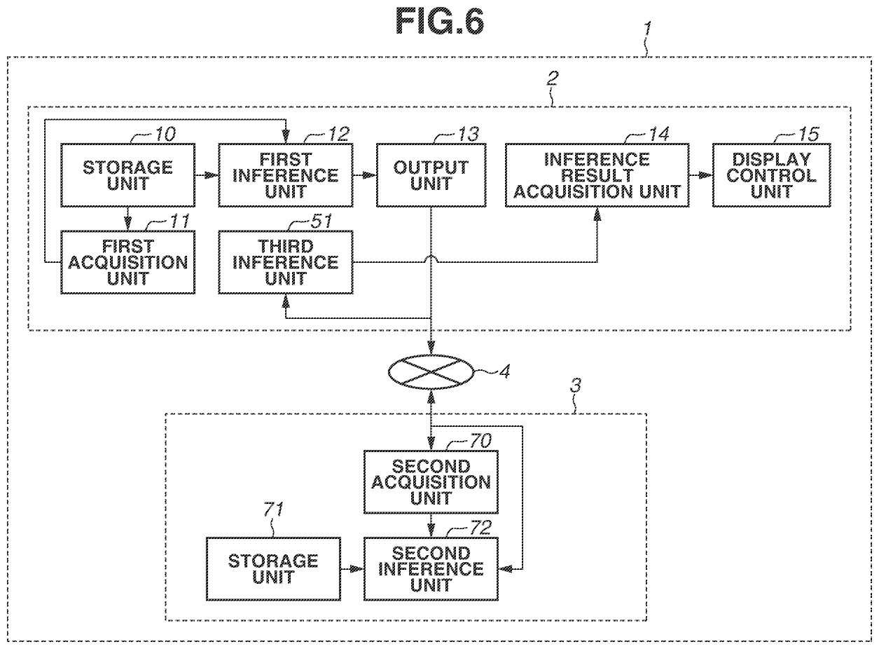

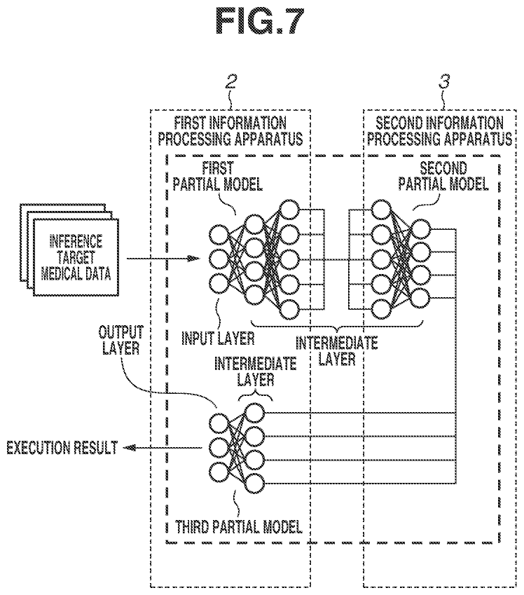

The first and second exemplary embodiments described above illustrate an example where the information processing system 1 includes the first information processing apparatus 2 including the first partial model that includes the input layer and at least some of the intermediate layers of the trained inference model based on the neural network including the input layer, the intermediate layers, and the output layer to perform inference processing on medical data and corresponds to a plurality of second partial models formed of layers different from the layers forming the first partial model, and the second information processing apparatus 3 including the plurality of second partial models formed of layers different from the layers forming the first partial model. In the third exemplary embodiment, a configuration example of the information processing system 1 in which the first information processing apparatus 2 further includes a third inference unit 51 that performs inference processing using a plurality of third partial models each including the output layer and information can be output to the first information processing apparatus 2 will be described with reference to FIG. 6. An inference model according to the present exemplary embodiment will be described with reference to FIG. 7.

In particular, differences between the third exemplary embodiment and the first and second exemplary embodiments will be described and redundant descriptions are omitted as appropriate.

Like in the first exemplary embodiment, the information processing system 1 according to the third exemplary embodiment includes the first information processing apparatus 2, the second information processing apparatus 3, and the network 4 that interconnects the first information processing apparatus 2 and the second information processing apparatus 3 in a communicable manner. The information processing system 1 includes three partial models.

The first information processing apparatus 2 that is operated by the user includes a first partial model including an input layer and at least some of intermediate layers in a trained inference model based on a neural network including the input layer, the intermediate layers, and an output layer to perform inference processing on medical data and corresponds to a plurality of second partial models formed of layers different from the layers forming the first partial model, and further includes third partial models each including some of the intermediate layers and the output layer. The third partial models are stored in association with the plurality of second partial models, respectively, and third inference processing is performed using, as an input, the output from the intermediate layers as a result of inference processing from the second partial models.

On the other hand, the second information processing apparatus 3 that is operated by a person who manages the inference model includes a plurality of second partial models including at least some of the intermediate layers between the intermediate layers of the first partial model and the intermediate layers of the third partial models in the intermediate layers of the inference model. The number of partial models is variable and the partial model including the input layer and the partial model including the output layer may be included in the first information processing apparatus 2 that is operated by the user authorized to manage the inference target medical data. Also, the number of the first information processing apparatuses 2 is not particularly limited, as long as each information processing apparatus 2 is managed by the user. The determination as to whether to perform inference processing using the (i+1)th second partial model based on the inference result of the i-th partial model as described in the second exemplary embodiment may be made by the second inference unit 72 that has received the output from the third inference unit 51. Alternatively, the third inference unit 51 may determine whether to perform inference processing using the (i+1)th partial model based on the output from the i-th third partial model corresponding to the i-th second partial model. In this case, the determination as to whether to perform inference processing by the second inference unit 72 or the third inference unit 51 is a determination on the second partial models that match the selection information and the third partial models corresponding to the second partial models, respectively.

The information processing system 1 according to the present exemplary embodiment can secure the confidentiality of the inference models for inference target medical data while protecting the privacy of the medical data, and can also secure the privacy of inference results because the inference results are output at the user side. The determination as to whether to perform inference processing using the (i+1)th partial model by the second inference unit 72 or the third inference unit 51 based on the inference result of the i-th partial model makes it possible to reduce the calculation cost by eliminating unnecessary partial models. Also, in the case of updating parameters for partial models and the like by a technique such as additional training to be described below, there is no need to transmit ground truth data for calculating a loss function to an external apparatus. For example, in the case of increasing the image quality in an inference model, if the output layer is included in an information processing apparatus that is not owned by the user, the second information processing apparatus 3 generates a high-quality image to represent the inference target medical data, which makes it difficult to protect the privacy of the medical data. For this reason, the first information processing apparatus 2 includes a partial model including the output layer and performs inference processing using the partial model, thereby making it possible to protect the privacy of the output information.

The information processing apparatus 1 according to according to the present exemplary embodiment includes at least the storage unit 10, the first acquisition unit 11, the first inference unit 12, the output unit 13, the inference result acquisition unit 14, the display control unit 15, and the third inference unit 51 that performs third inference processing using third partial models respectively corresponding to the plurality of second partial models described above.

The information processing apparatus 2 according to the present exemplary embodiment includes the storage unit 71 and the second inference unit 72. Each of the plurality of second partial models used in the second inference unit 72 includes a network formed of intermediate layers located between the intermediate layers of the first partial model and the intermediate layers of the third partial models in the trained inference model.

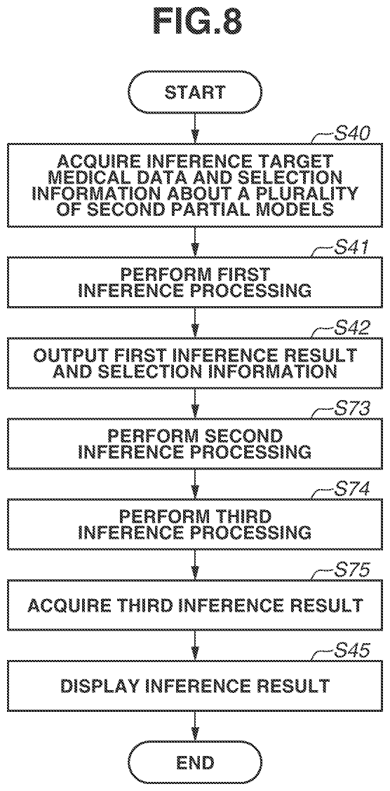

An inference process according to the present exemplary embodiment will be described below with reference to FIG. 8.

Steps S40 to S42 are similar to those of the first exemplary embodiment, and thus the descriptions thereof are omitted.

In step S73, the second information processing apparatus 3 receives, as an input, the inference result from the first inference unit 12 and performs second inference processing using a plurality of second partial models. Each of the plurality of second partial models includes a network formed of intermediate layers located between the intermediate layers of the first partial model and the intermediate layers of the third partial models in the trained inference model. The second inference unit 72 transmits the output from the intermediate layers in the second partial models to the first information processing apparatus 2 as the second inference result. Then, the processing proceeds to step S74.

In step S74, the third inference unit 51 in the first information processing apparatus 2 receives, as an input, the second inference result and performs third inference processing using a plurality of third partial models. The third inference unit 51 transmits the third inference result to the inference result acquisition unit 14. Then, the processing proceeds to step S75.

In step S75, the inference result acquisition unit 14 acquires the third inference result as the inference result corresponding to the inference target medical data, and transmits the acquired inference result to the display control unit 15.

The description of step S45 is omitted. The inference results to be displayed on the display device 26 are inference results obtained by performing a series of inference processing (first inference processing, second inference processing, and third inference processing) on the inference target medical data by the first information processing apparatus 2 and the second information processing apparatus 3.

According to the present exemplary embodiment, it is possible to perform the inference processing by securing the confidentiality of the inference model for medical data while protecting the privacy of the inference target medical data, and also protecting the privacy of the plurality of inference results.

Inference model training processing according to the fourth and fifth exemplary embodiments will be described below. The inference model used in the above-described inference processing is not limited to an inference model generated by performing the training processes according to the fourth and fifth exemplary embodiments. The inference model used in the following exemplary embodiments may be an inference model obtained without performing training processing, or may be a trained inference model obtained by performing training processing.

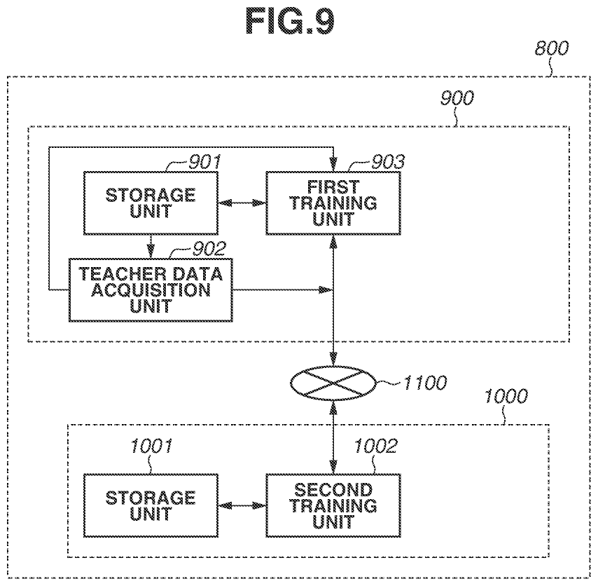

A configuration example of an information processing system 800 according to the fourth exemplary embodiment will be described with reference to FIG. 9. The information processing system 800 includes a first information processing apparatus 900 as an information processing apparatus that is operated by the user of the inference model, a second information processing apparatus 1000 as an information processing apparatus that is operated by the provider of the inference model, and a network 1100 that interconnects the first information processing apparatus 900 and the second information processing apparatus 1000. A network configuration of the inference model corresponding to each of the first information processing apparatus 900 and the second information processing apparatus 1000 will be described with reference to FIG. 9.

The first information processing apparatus 900 includes a first partial model including an input layer and at least some of intermediate layers of an inference model based on a neural network including the input layer, the intermediate layers, and an output layer to perform inference processing on medical data.

The second information processing apparatus 1000 includes a plurality of second partial models formed of layers different from the layers forming the first partial model in the inference model. In the present exemplary embodiment, each of the second partial models includes some of the intermediate layers and the output layer. Thus, the first partial model including the input layer is provided in the first information processing apparatus 900 that is operated by the user and the plurality of second partial models, which is a part of the inference model, is provided in the second information processing apparatus 1000 that is operated by the provider of the inference model, thereby making it possible to perform inference model training processing by securing the confidentiality of the inference model while protecting the privacy of the medical data. In this case, the first partial model may be formed as a public network and the second partial models may be formed as a private network. The provider of the inference model uses the second partial models as the private network, thereby further enhancing the confidentiality of the inference model.

The first information processing apparatus 900 includes a storage unit 901 that stores training data and information about the inference model. The first information processing apparatus 900 also includes a training data acquisition unit 902 that acquires training data from the storage unit 901, and a first training unit 903 that learns the first partial model based on the acquired training data. The storage unit 901 may be composed of a storage device or the like managed by the user of the inference model. Upon completion of training processing on the first partial model, the first training unit 903 stores information about the trained first partial model in the storage unit 901. If the first partial model corresponds to the plurality of second partial models, the parameters for the second partial models are trained and updated without updating the parameter for the first partial model on which training processing is completed, thereby generating the plurality of second partial models corresponding to a plurality of tasks or classes, respectively.

The second information processing apparatus 1000 includes a storage unit 1001 that stores inference model information, and a second training unit 1002 that learns the second partial models.

Training processing to be performed by each training unit refers to processing for propagating training data forward to a partial model and updating the parameter of the partial model using error information acquired by a back-propagation method. The training data is composed of training data and a ground truth label. The training data is, for example, medical data. The ground truth label is information indicating an object captured in the medical data. The ground truth label may be set as ground truth data indicating the content of a captured object for each pixel. The first information processing apparatus 900 transmits model selection information to the second information processing apparatus 1000, thereby making it possible to select an appropriate model even in a case where at least a plurality of first partial models or a plurality of second partial models is present.

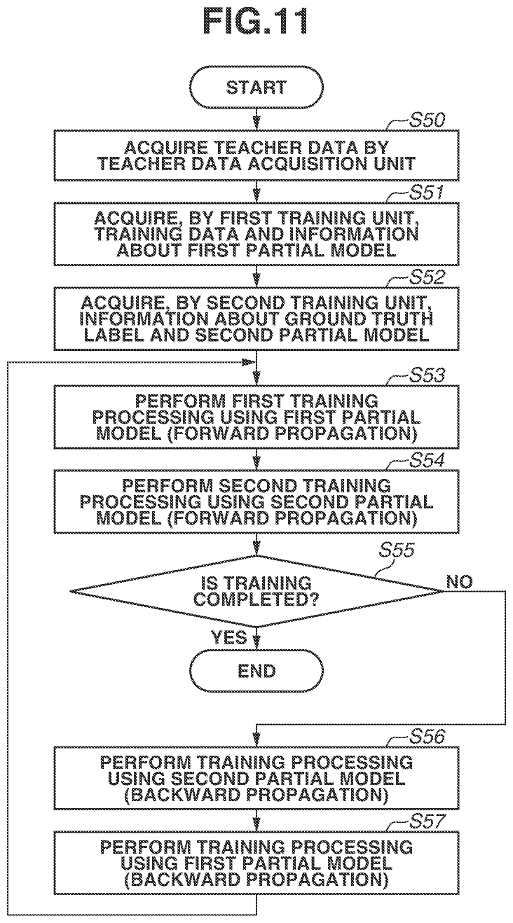

An example of training processing to be performed by the information processing system 800 according to the present exemplary embodiment will be described below with reference to a flowchart illustrated in FIG. 11.

In step S50, the training data acquisition unit 902 acquires training data as a pair of training data and a ground truth label from the storage unit 901. The training data acquisition unit 902 transmits information about the training data to the first training unit 903, and transmits the ground truth label to the second information processing apparatus 1000. Then, the processing proceeds to step S51.

In step S51, the first training unit 903 acquires the training data transmitted from the training data acquisition unit 902 and also acquires information about the first partial model from the storage unit 901. The first training unit 903 may transmit information indicating the acquired first partial model to the second information processing apparatus 1000.

In step S52, the second training unit 1002 acquires information about the second partial models from the storage unit 1001 and also acquires information about the ground truth label from the training data acquisition unit 902.

In step S53, the first training unit 903 inputs and propagates the training data forward to the first partial model, and then performs first training processing as a part of the training process. Upon completion of the first training processing, the first training unit 903 transmits data such as tensors generated in the first training processing to the second training unit 1002.

In step S54, the second training unit 1002 inputs and propagates the parameter transmitted from the first training unit 903 forward to the second partial model, and then performs second training processing as a part of the training process.

In step S55, the second training unit 1002 compares the output from the second partial models by forward propagation of the second partial models including the output layer in the network configuration with the ground truth label, and acquires error information using the loss function. The second training unit 1002 determines whether the training processing is completed. The second training unit 1002 determines whether the training processing is completed based on, for example, whether the calculated error information indicates a value less than a predetermined value, or whether training processing has been performed a predetermined number of times. If the second training unit 1002 determines that the training processing is completed (YES in step S55), the processing flow ends. On the other hand, if the second training unit 1002 determines that the training processing is continued (NO in step S55), the processing proceeds to step S56. The determination in step S55 may be made by the first training unit 903 before the first training processing is started.

In step S56, the second training unit 1002 updates the parameters for the second partial models based on the error information calculated in step S55. Each parameter indicates, for example, a weight or bias. The second training unit 1002 transmits, to the input layer, the error information from the intermediate layer closest to the output layer by a back-propagation method. The second training unit 1002 transmits the error information from the intermediate layer closest to the input layer forming the second partial model to the first training unit 903. Then, the processing proceeds to step S57.

In step S57, the first training unit 903 updates the parameter for the first partial model based on error information transmitted from the second training unit 1002. When the parameter for the first partial model is updated, the processing proceeds to step S53. The process of updating the parameter can be omitted. As described above, the parameter for the first partial model may be fixed during training, and only the parameters for the second partial models may be trained. Examples of the first partial model include a trained inference model generated by training the inference model trained to classify the classes correlated with the plurality of second partial models, or any one of the plurality of second partial models. As described above in step S55, the determination as to whether the training processing is completed may be made by the first training unit 903 at this timing.

The information processing system 800 according to the present exemplary embodiment configured as described above can perform training processing using a plurality of inference models while protecting the privacy of medical data and securing the confidentiality of the inference model. The number of partial models to be used is not limited to two. If the partial model including the input layer is included in the information processing apparatus that is operated by the user of the inference model, the training processing according to the present exemplary embodiment is applicable. If a plurality of inference models is used, information for selecting the partial models corresponding to the plurality of inference models, respectively, may be transmitted from the first training unit 903 to the second training unit 1002. The partial models may be selected by the user, or may be selected by the information processing apparatus depending on input data.

The fourth exemplary embodiment described above illustrates an example where training processing is performed in the information processing system 800 in which the first information processing apparatus 900 includes the first partial model including the input layer and at least some of the intermediate layers and the second information processing apparatus 1000 includes the second partial models formed of layers different from the layers of the first partial model.

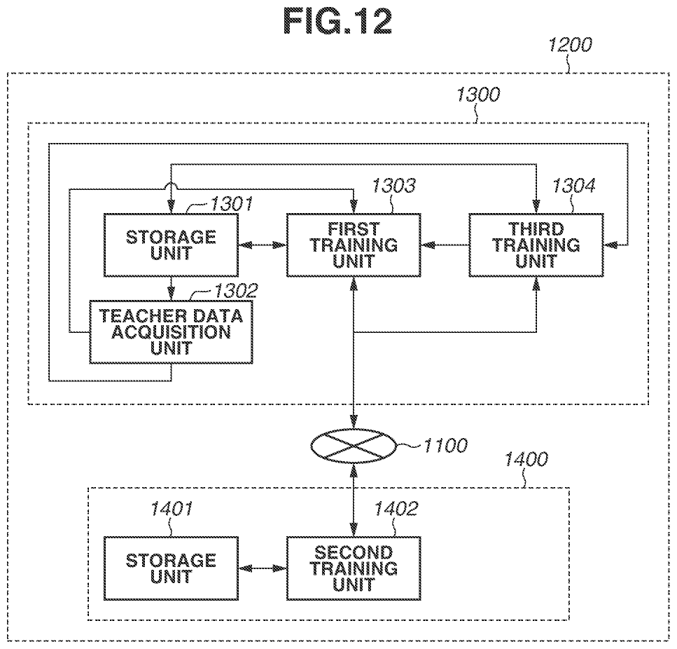

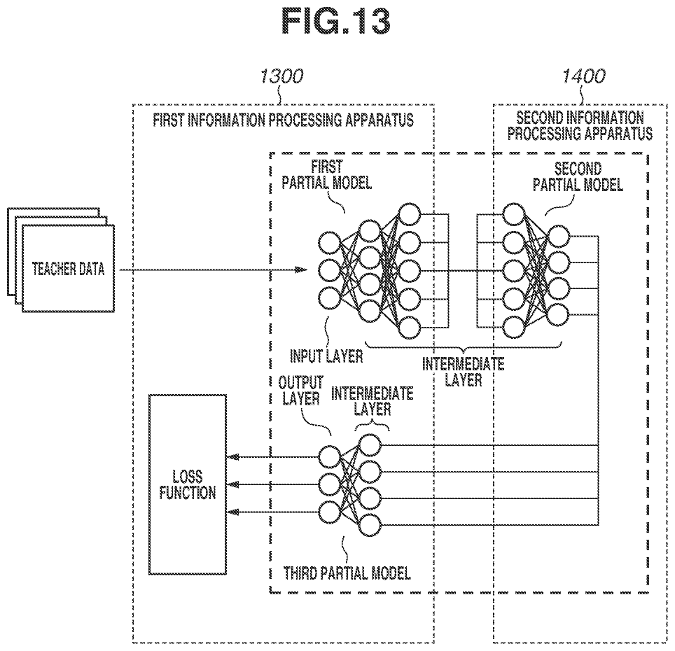

In the fifth exemplary embodiment, training processing to be performed by an information processing system in which a first information processing apparatus includes third partial models each including at least intermediate layers different from the intermediate layers of the first partial model and the intermediate layers of the second partial model and an output layer will be described with reference to FIG. 12. A network configuration example of the inference model according to the fifth exemplary embodiment will be described with reference to FIG. 13. The descriptions of components of the fifth exemplary embodiment that are similar to those of the third exemplary embodiment are omitted as appropriate.

An information processing system 1200 according to the present exemplary embodiment includes a first information processing apparatus 1300 as an information processing apparatus that is operated by the user of the inference model, a second information processing apparatus 1400 as an information processing apparatus that is operated by the provider of the inference model, and the network 1100 that interconnects the first information processing apparatus 1300 and the second information processing apparatus 1400.

The first information processing apparatus 1300 includes a first partial model that includes an input layer and at least some of intermediate layers of an inference model based on a neural network including the input layer, the intermediate layers, and an output layer to perform inference processing on medical data. The first information processing apparatus 1300 further includes third partial models each including at least the output layer in the inference model.

The second information processing apparatus 1400 includes second partial models each including at least some of the intermediate layers of the inference model.

In the configuration according to the present exemplary embodiment, the first information processing apparatus 1300 includes the third partial models further including the output layer, thereby making it possible to perform training processing on the inference model without the need of transmitting the training data and ground truth label that constitute the training data to the second information processing apparatus 1400. In addition, the provision of the second partial models including at least some of the intermediate layers forming the inference model enables the provider of the inference model to secure the confidentiality of the inference model. According to the present exemplary embodiment, the number of partial models, the number of information processing apparatuses, and the like can be appropriately set as long as the partial model including the input layer and the partial model including the output layer are included in the first information processing apparatus 1300.

The first information processing apparatus 1300 includes a storage unit 1301 that stores training data and information about the inference model. The first information processing apparatus 1300 also includes a training data acquisition unit 1302, a first training unit 1303, and a third training unit 1304. The training data acquisition unit 1302 acquires training data from the storage unit 1301. The first training unit 1303 learns the first partial model based on the acquired training data. The third training unit 1304 learns the third partial models.

The second information processing apparatus 1400 includes a storage unit 1401 that stores information about the inference model, and a second training unit 1402 that learns the second partial models.

In this case, the training processing refers to a series of processing for propagating the training data constituting the training data forward to a partial model and updating the parameter for the partial model by performing back-propagation of error information based on a ground truth label and an output value from the output layer (back-propagation method). The training data is composed of training data and a ground truth label.

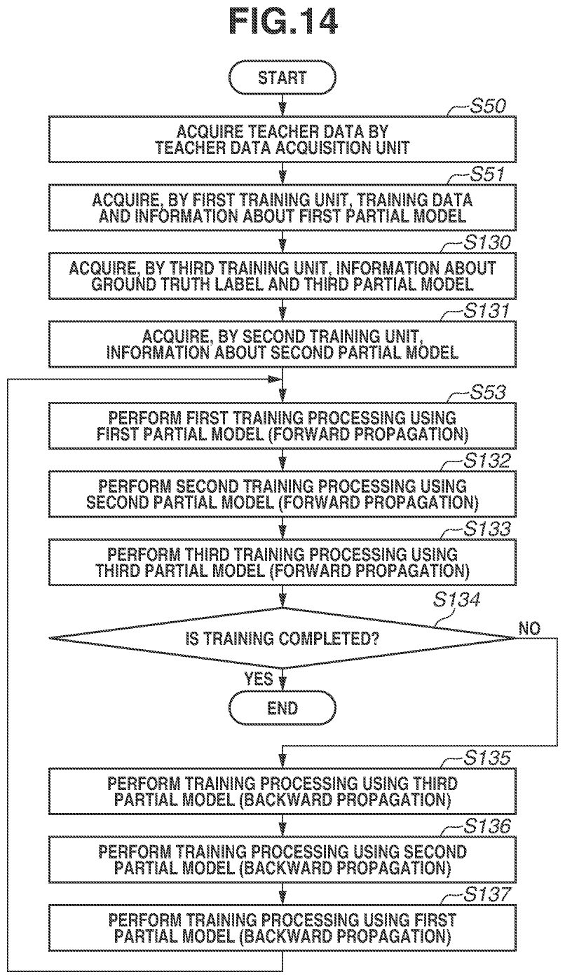

An example of the training process to be performed by the information processing system 800 according to the present exemplary embodiment will be described below with reference to a flowchart illustrated in FIG. 14. Only differences from the flowchart illustrated in FIG. 11 will be described.

In step S130, the third training unit 1304 acquires information about the third partial models from the storage unit 1301 and also acquires information about the ground truth label from the training data acquisition unit 1302.

In step S132, the second training unit 1402 inputs and propagates the parameter transmitted from the first training unit 1303 forward to the second partial models, and then performs second training processing as a part of the training process.

In step S133, the third training unit 1304 inputs and propagates the parameter transmitted from the second training unit 1402 forward to the third partial models, and then performs third training processing as a part of the training process.

In step S134, the third training unit 1304 compares the output from the third partial models by forward propagation of the third partial models including the output layer in the network configuration with the ground truth label, and acquires error information using the loss function. The third training unit 1304 determines whether the training processing is completed. The third training unit 1304 determines whether the training processing is completed based on, for example, whether the calculated error information indicates a value less than a predetermined value, or whether training processing has been performed a predetermined number of times. If the third training unit 1304 determines that the training processing is completed (YES in step S134), the processing flow ends. On the other hand, if the third training unit 1304 determines that the training processing is to be continued (NO in step S134), the processing proceeds to step S135. The determination as to whether the training processing is completed in step S134 may be made by the first training unit 1303 before the first training processing is started.

In step S135, the third training unit 1304 updates the parameters for the third partial models based on the error information calculated in step S134. Each parameter indicates, for example, a weight or bias. The third training unit 1304 transmits the error information from the intermediate layer closest to the output layer to the input layer by back-propagation. The third training unit 1304 transmits the error information from the intermediate layer closest to the input layer forming the third partial models to the second training unit 1402. Then, the processing proceeds to step S136.

In step S136, the second training unit 1402 updates the parameters for the second partial models based on the error information transmitted from the third training unit 1304. The second training unit 1402 transmits the error information from the intermediate layer closest to the output layer to the input layer by back-propagation and transmits the output from the intermediate layer close to the input layer to the first training unit 1303. Then, the processing proceeds to step S137.

In step S137, the first training unit 1303 updates the parameter for the first partial model based on the error information transmitted from the second training unit 1402. As described above, the parameter for the first partial model is trained to correspond to the plurality of second partial models. Accordingly, a parameter generated by specific training processing may be fixed as the parameter for the first partial model. The specific training processing refers to a trained inference model generated by training the inference model trained to classify the classes correlated with the plurality of third partial models, or any one of the plurality of third partial models. After completion of updating of the parameter for the first partial model or back-propagation of the error information with the fixed parameter, the processing proceeds to step S53. As described above in step S134, the determination as to whether the training processing is completed may be made by the first training unit 1303 at this timing.

The information processing system 1200 according to the present exemplary embodiment configured as described above can perform training processing using a plurality of inference models while protecting the privacy of medical data and securing the confidentiality of the inference models. In addition, there is no need to transmit the training data and ground truth label that constitute the training data from the information processing apparatus that is operated by the user of the inference model, thereby making it possible to further secure the confidentiality of medical data.

Inference models trained according to the fourth and fifth exemplary embodiments may be used as inference models for inference processing according to the first and second exemplary embodiments. The training processing according to the fourth and fifth exemplary embodiments is also effective as a technique for additional training of the inference model for inference processing.

### Modified Example 1

The training processing to be performed in a case where the partial model including the output layer is included in the first information processing apparatus and the training processing to be performed in a case where the partial model including the output layer is included in the second information processing apparatus have been described above.

In Modified Example 1, a configuration for setting the partial model including the output layer in the information processing apparatus that is operated by the user of the inference model or in the information processing apparatus that is operated by the provider of the inference model depending on the output of the inference model will be described.

For example, in a case where the output of the inference model is equivalent to input training data indicating, for example, input data with a higher resolution, the inference model including the output layer is located in the information processing apparatus that is operated by the user of the inference model. On the other hand, if the inference model for performing class classification or detection processing on medical data is output, the inference model including the output layer is located in the information processing apparatus that is operated by the provider of the inference model. Each partial model is configured depending on the output of the inference model, and thus training processing can be performed while balancing machine resources and the confidentiality of medical data. The configuration of each partial model depending on the output of the inference model is also effective for inference processing. For example, the first partial model for performing first inference processing may be selected from among a plurality of first inference models depending on inference target medical data, and second inference models for performing second inference processing may be selected from among a plurality of second partial models depending on inference target medical data.

### Modified Example 2

The inference model training processing performed by the back-propagation method has been described above.

In Modified Example 2, inference model training processing to be performed by a training method other than the back-propagation method will be described.

For example, a method of training a model to estimate a gradient to be obtained for each layer, such as Synthetic Gradient, a method using a fixed random matrix during back-propagation, such as Feedback Alignment, a method of propagating a target instead of an error, such as Target Prop, and any other method may be used.

### Other Examples

The present invention can also be implemented by executing the following processing. That is, software (program) for implementing the functions according to the exemplary embodiments described above is supplied to a system or an apparatus via a network or various storage media, and a computer (or a CPU, a microprocessor unit (MPU), etc.) in the system or the apparatus reads out the program and executes the program.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)?), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-112966, filed Jul. 7, 2021, which is hereby incorporated by reference herein in its entirety.

What is claimed is:

1. An information processing system comprising:
a first information processing apparatus; and
a second information processing apparatus configured to communicate with the first information processing apparatus via a network,
wherein the first information processing apparatus includes:
a first acquisition unit configured to acquire inference target medical data and selection information indicating a partial model to be applied to the inference target medical data;
a first inference unit configured to perform first partial inference processing on the inference target medical data using a first partial model, the first partial model including an input layer and at least some of intermediate layers of a neural network, wherein a plurality of second partial models includes the intermediate layers not included in the first partial model, and the neural network includes the input layer, the intermediate layers, and an output layer; and
a first output unit configured to output a result of the first inference processing and the selection information to the second information processing apparatus, and
wherein the second information processing apparatus includes:
a second acquisition unit configured to acquire the result of the first inference processing and the selection information from the first information processing apparatus; and
a second inference unit configured to perform second inference processing by inputting the result of the first inference processing to a second partial model selected from among the plurality of second partial models based on the selection information.

2. The information processing system according to claim 1, wherein each of the plurality of second partial models is a partial model generated by additional training with a fixed parameter for the first partial model.

3. The information processing system according to claim 1, wherein the second partial models are partial models in which at least one of an inference task to be performed by each second partial model and an inference class of each second partial model is different from that of other second partial model.

4. The information processing system according to claim 1, wherein the selection information is information including an application order of the plurality of second partial models.

5. The information processing system according to claim 4, wherein the second inference unit performs inference processing using a predetermined second partial model in the plurality of second partial models based on the application order and determines whether to perform inference processing using other partial model depending on a result of the inference processing.

6. The information processing system according to claim 5, wherein the second inference unit determines not to perform inference processing using other partial model depending on the result of the inference processing.

7. The information processing system according to claim 1, wherein the output unit outputs an output from the intermediate layers forming the first partial model to the second information processing apparatus.

8. The information processing system according to claim 1,
wherein the first information processing apparatus further includes an inference result acquisition unit, and
wherein the inference result acquisition unit acquires an inference result from the second inference unit.

9. The information processing system according to claim 1, wherein in the second information processing apparatus, the plurality of second partial models includes a second partial model including the output layer, and the second inference unit performs the second inference processing using the second partial model.

10. The information processing system according to claim 5, wherein the second inference unit further inputs the result of the inference processing to other second partial model.

11. An information processing apparatus comprising:
an acquisition unit configured to acquire, from another information processing apparatus, a result of first inference processing on inference target medical data using a first partial model including an input layer and at least some of intermediate layers of a neural network, wherein a plurality of second partial model includes the intermediate layers not included in the first partial model, and the neural network includes the input layer, the intermediate layers, and an output layer; and
a second inference unit configured to perform second inference processing by inputting a result of the first inference processing to a second partial model of the plurality of second partial models selected based on selection information.

12. An information processing method comprising:
acquiring, from another information processing apparatus, a result of first inference processing on inference target medical data using a first partial model including an input layer and at least some of intermediate layers of a neural network, wherein a plurality of second partial models includes the intermediate layers not included in the first partial model, and the neural network includes the input layer, the intermediate layers, and an output layer; and
performing second inference processing by inputting a result of the first inference processing to a second partial model of the plurality of second partial models selected based on selection information.

13. A non-transitory storage medium storing a program that causes a computer to execute an information processing method according to claim 12.
